(19) United States
(12) Patent Application Publication
(10) Pub. No.: US 2006/0228387 A1
(43) Pub. Date: Oct. 12, 2006

(54) Dihydronepetalactams and N-substituted derivatives thereof

(76) Inventors: Mark A. Scialdone, West Grove, PA (US); Ann Y. Liauw, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/314,385

(22) Filed: Jun. 16, 2006

Related U.S. Application Data
(60) Provisional application No. 60/640,130, filed on Dec. 29, 2004.
(60) Provisional application No. 60/640,129, filed on Dec. 29, 2004.

(51) Int. Cl. A01N 25/00; A01N 43/42; C07D 221/02; A01N 65/00

(52) U.S. Cl. 424/405; 514/300; 546/112; 424/725; 424/756; 424/745; 424/764; 424/735; 424/736; 424/747; 424/765

(57) ABSTRACT

Dihydronepetalactams and N-substituted derivatives thereof are prepared by alkylation of metallated lactams. Dihydronepetalactams and N-substituted derivatives thereof have utility as insect repellents.

This application claims the benefit of U.S. Provisional Application No. 60/640,129, filed Dec. 29, 2004, and U.S. Provisional Application No. 60/640,130, filed Dec. 29, 2004, each of which is incorporated in its entirety as a part hereof for all purposes.

### TECHNICAL FIELD

The present invention is directed to dihydronepetalactams and N-substituted derivatives thereof, which are useful as repellents for insects and arthropods.

### BACKGROUND

Insect repellents are used globally as a means of reducing human-insect vector contact, thereby minimizing the incidence of vector-borne disease transmission as well as the general discomfort associated with insect bites. The best known and most widely used active ingredient in commercial topical insect repellents is the synthetic benzene derivative, N,N-diethyltoluamide (DEET).

Nepetalactone (represented in general schematically by Formula II), a major component of an essential oil secreted by plants of the genus Nepeta and the active ingredient in catnip, is known to be an effective, natural repellent to a variety of insects [Eisner, T., Science (1964) 146:1318-1320]. 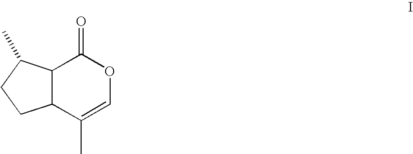
U.S. Pat. No. 6,524,605 discloses the repellency of nepetalactone, as well as the individual cis,trans (Z,E) and trans,cis (E,Z) isomers, against German cockroaches.

Dihydronepetalactone (DHN), represented schematically by Formula I, a chemical which is secreted by certain insects, is known to exhibit insect repellency activity. 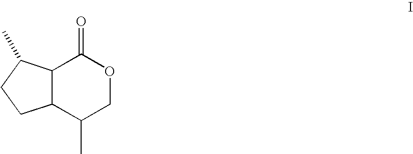
Jefson et al [J. Chemical Ecology (1983) 9:159-180] described the repellent effect of DHN on feeding by ants of the species Monomorium destructor. More recently, Hallahan (WO 2003/079786) has found that DHN compares favorably as an insect repellent with DEET.

A need remains, however, for the continued availability of as wide a variety of insect repellents as possible, and it has been found that dihydronepetalactams, and derivatives thereof, are useful as repellents for insects and arthropods.

### SUMMARY

In one embodiment, this invention relates to a compound represented schematically in general by Formula (IV): 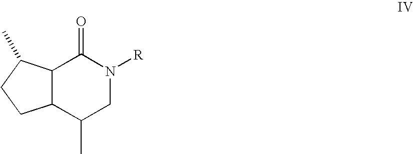
wherein R is (1) an alkane radical other than methyl, (2) an alkene radical, (3) an alkyne radical, or (4) an aromatic radical.

Another embodiment of this invention is a composition of matter that includes (a) a carrier, and (b) a compound described generally as above in Formula IV, wherein R is H, an alkane radical, an alkene radical, an alkyne radical, or an aromatic radical.

A further embodiment of this invention is a method for repelling an insect or arthropod by exposing the insect or arthropod to a compound described generally as above in Formula IV, wherein R is H, an alkane radical, an alkene radical, an alkyne radical, or an aromatic radical.

Yet another embodiment of this invention is the use of a compound described generally as above in Formula IV, wherein R is H, an alkane radical, an alkene radical, an alkyne radical, or an aromatic radical to repel insects and/or arthropods from a human, animal or inanimate host.

Yet another embodiment of this invention is an article of manufacture that incorporates a compound described generally as above in Formula IV, wherein R is H, an alkane radical, an alkene radical, an alkyne radical, or an aromatic radical.

Yet another embodiment of this invention is a method of fabricating an insect repellent composition, or an insect repellent article of manufacture, by forming the composition from, or incorporating into the article, a compound described generally as above in Formula IV, wherein R is H, an alkane radical, an alkene radical, an alkyne radical, or an aromatic radical

Yet another embodiment of this invention is a method of fabricating a composition to be applied to skin, or a fragrant article of manufacture, by forming the composition from, or incorporating into the article, a compound described generally as above in Formula IV, wherein R is H, an alkane radical, an alkene radical, an alkyne radical, or an aromatic radical. The composition to be applied to skin may have fragrant or other therapeutic properties.

### BRIEF DESCRIPTION OF THE DRAWINGS

FIGS. 1-10 represent the results of testing the indicated dihydronepetalactam or derivative compounds, and/or compositions thereof, against the indicated controls for their effect on the probing behavior of Aedes aegypti mosquitoes in the in vitro Gupta box landing assay procedure, described herein. The horizontal scale shows time in minutes, and the vertical scale shows mean number of landings of mosquitoes.

### DETAILED DESCRIPTION

This invention relates to novel compounds based on C₂ to C₂₀ N-substituted dihydronepetalactams, which are useful as insect repellents. The present invention also relates to dihydronepetalactams and N-substituted dihydronepetalactams, and compositions thereof, which are also useful as insect repellents.

This invention provides novel compounds that may be represented schematically by the structure of Formula IV, 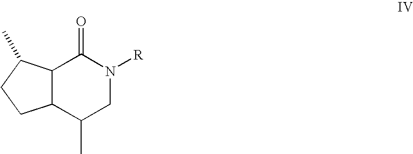
wherein R is (1) an alkane radical other than methyl, (2) an alkene radical, (3) an alkyne radical, or (4) and aromatic radical. The term “alkane” refers to a saturated hydrocarbon having the general formula CₙH₂ₙ₊₂. The term “alkene” refers to an unsaturated hydrocarbon that contains one or more C═C double bonds, and the term “alkyne” refers to an unsaturated hydrocarbon that contains one or more carbon-carbon triple bonds. An alkene or alkyne requires a minimum of two carbons. A cyclic compound requires a minimum of three carbons. The term “aromatic” refers to benzene and compounds that resemble benzene in chemical behavior.

While there is in principle no limitation on the type of alkanyl, alkenyl, alkynyl or aromatic groups that are useful as values for R in the practice of the invention, there will be practical considerations as to the size of the R substituent that would have practical use in commerce. Furthermore, it may be desirable to avoid incorporating highly reactive functionality in the R substituents to avoid side reactions.

Preferably, R of Formula (IV) is (1) C₂ to C₂₀ alkane, (2) C₂ to C₂₀ alkene, (3) C₃ to C₂₀ alkyne, or (4) C₆ to C₂₀ aromatic. More preferably, R of Formula (IV) is selected from the group consisting of (1) C₂H₅; (2) C₃ to C₂₀ straight-chain, branched or cyclic alkane or alkene; (3) C₃ to C₂₀ straight-chain, branched or cyclic alkane or alkene comprising a heteroatom selected from the group consisting of O, N and S; (4) unsubstituted or substituted C₆ to C₂₀ aromatic, wherein the substituent is selected from the group consisting of (a) C₁ to C₁₂ straight-chain, branched or cyclic alkane or alkene, optionally substituted with Cl, Br or F, and (b) a halogen selected from the group consisting of Cl, Br and F; and (5) unsubstituted or substituted C₆ to C₂₀ aromatic comprising a heteroatom selected from the group consisting of O, N and S, wherein the substituent is selected from the group consisting of (a) C₁ to C₁₂ straight-chain, branched or cyclic alkane or alkene, optionally substituted with Cl, Br or F, and (b) a halogen selected from the group consisting of Cl, Br and F.

In another embodiment, R is selected from the group consisting of (1) C₂H₅; (2) C₃ to C₁₂ straight-chain, branched or cyclic alkane or alkene; and (3) C₃ to C₁₂ straight-chain, branched or cyclic alkane or alkene comprising a heteroatom selected from the group consisting of O, N and S. In another more specific embodiment, R may be unsubstituted or substituted phenyl, wherein the substituent is selected from the group consisting of (a) C₁ to C₁₂ straight-chain, branched or cyclic alkane or alkene, optionally substituted with Cl, Br or F, and (b) a halogen selected from the group consisting of Cl, Br and F. An example of an alkane substituted with F is CF₃.

Particularly preferred values for R include ethyl, n-propyl, i-propyl, n-butyl, t-butyl, n-pentyl, n-hexyl, cyclohexyl, n-octyl, trimethylpentyl, cyclooctyl, allyl, propargyl, phenyl, methylphenyl, ethylphenyl, n-propylphenyl, n-butylphenyl, t-butylphenyl, p-chlorophenyl, and p-bromophenyl.

The compounds represented by Formula IV may be prepared by alkylation of nepetalactam, followed by hydrogenation, or by alkylation of dihydronepetalactam. Nepetalactam may be prepared from nepetalactone. The nepetalactone bicyclic structure can exist in any of four stereoisomeric forms, as shown in the structures of Formulae IIa-IId.

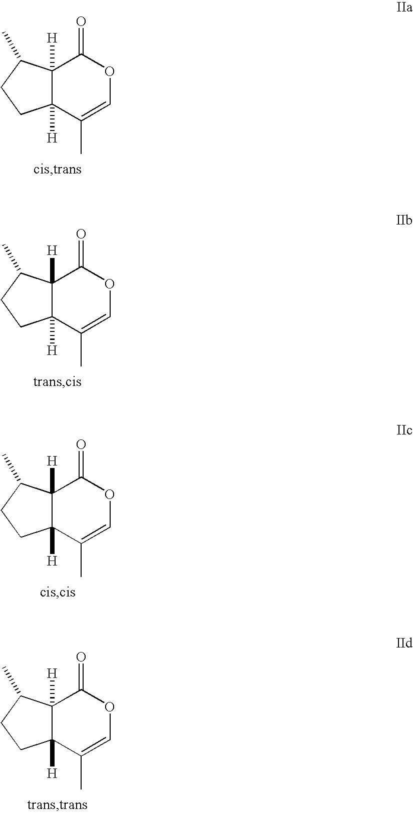

Nepetalactone extracted from the essential oil of the Nepeta (catmint) plant leaves is a preferred source of raw material as nepetalactone is present in large quantity therein and may be readily purified therefrom. This produces a desirable route from a natural product to the compounds of the invention. Fractional distillation, as described herein, has been found to be an effective method for both purifying nepetalactone from the essential oils, and for separating the several stereoisomers from one another. Chromatographic separations are also suitable.

Only the first three listed stereoisomers of nepetalactone exist in the essential oil of the Nepeta cateria plant. Cis,trans-nepetalactone is the predominant isomer that may be isolated from the Nepeta cateria plant and is therefore the most useful because of availability. Other plant species have been identified of which the essential oils are enriched with the trans,cis- and cis,cis-nepetalactone isomers.

Lactams are the nitrogen analogs of cyclic esters or lactones, and lactams, especially N-substituted lactams, are generally more stable to hydrolysis than their lactone counterparts. The synthesis of nepetalactam was demonstrated by Eisenbraun et al [J. Org. Chem. (1988) 53:3968-3972]. According to this method, nepetalactone (Formula III) was converted to nepetalactam (Formula III) in the presence of anhydrous ammonia (see Reaction I). 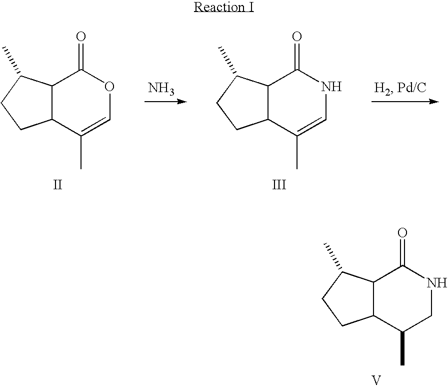
Nepetalactam was subsequently converted to dihydronepetalactam by hydrogenation in the presence of Pd/C as catalyst.

Methyl-substituted nepetalactam (IIIa) was synthesized by Eisenbraun et al (supra) using nepetalactone and methylamine, as shown in Reaction II. 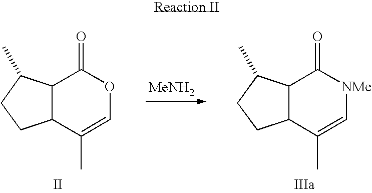
N-Methyl nepetalactam (IIIa) was then hydrogenated in the presence of a Pd/C catalyst to yield N-methyl dihydronepetalactam.

An alternative approach used by Eisenbraun et al (supra) to synthesize N-methyl dihydronepetalactam involved alkylating dihydronepetalactam using KOH, tetrabutylammonium bromide and methyl iodide.

Nepetalactam may thus be prepared by contacting cis,trans-nepetalactone (Formula II) with anhydrous ammonia according to the method described by Eisenbraun et al (supra), shown in Reaction III. 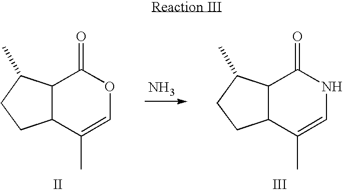
The use of cis,trans-nepetalactone is preferred as the starting material. Trans,cis-nepetalactone may be used but the resulting configuration of the N-substituted nepetalactam product is cis, trans due to epimerization of the stereochemical configuration at the bridgehead carbon next to the carbonyl to the cis, trans configuration.

N-Substituted dihydronepetalactams are synthesized by hydrogenation of nepetalactam to dihydronepetalactam followed by alkylation of the lactam nitrogen, or by alkylation of nepetalactam followed by hydrogenation of the N-substituted nepetalactam as shown in Reactions IV and V, respectively, below.

Hydrogenation of nepetalactams may be effected in the presence of a suitable active metal hydrogenation catalyst. Acceptable solvents, catalysts, apparatus and procedures for hydrogenation in general can be found in Augustine, Heterogeneous Catalysis for the Synthetic Chemist, Marcel Decker, New York, N.Y. (1996). The hydrogenation reaction may be carried out as described by Eisenbraun et al (supra), wherein N-methyl-3,4-dihydronepetalactam was treated with hydrogen in the presence of 10% Pd/C catalyst. The hydrogenation reaction may also be carried out according to the methods taught in WO 2003/084946 for the hydrogenation of nepetalactone, which is incorporated in its entirety as a part hereof for all purposes. Suitable methods of hydrogenation are also described in sources such as U.S. Pat. Nos. 6,664,402, 6,673,946, and 6,686,310.

N-Substituted dihydronepetalactams may be formed as shown in Reaction IV by reacting dihydronepetalactam (Formula V) with an appropriate metal hydride to form a dihydronepetalactam salt, followed by contacting the dihydronepetalactam salt with an appropriate alkylating agent to form the N-substituted dihydronepetalactam (Formula IV).

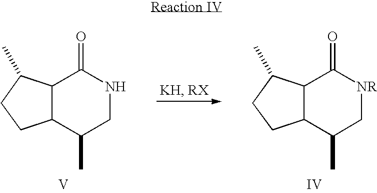

Alternatively, N-substituted dihydronepetalactams may be formed as shown in Reaction V by alkylation of nepetalactam, followed by hydrogenation of the N-substituted nepetalactam.

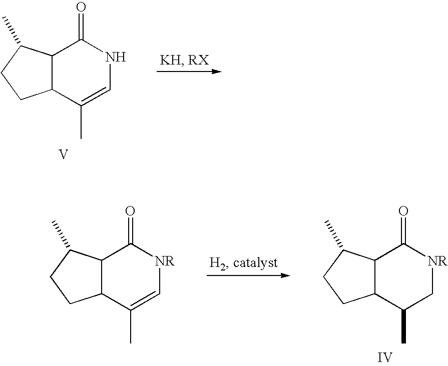

The conversion of dihydronepetalactam to N-substituted dihydronepetalactam is carried out at a temperature of from about 0° C. to about room temperature (about 25° C.). Similarly, the conversion of nepetalactone to N-substituted dihydronepetalactam is carried out at a temperature of from about 0° C. to about room temperature.

Metal hydrides are used to generate the amide-metal salt of dihydronepetalactam. Suitable metal hydrides include, but are not limited to, potassium hydride and sodium hydride. Very reactive metal hydrides such as lithium aluminum hydride, which would reduce the carbonyl group on the lactam, may be too reactive and are therefore less preferred.

Alkylating agents suitable for N-alkylation of the dihydronepetalactam salt include alkanyl, alkenyl, alkynyl or aryl chlorides, bromides, iodides, sulfates, mesylates, tosylates and triflates. Alkanyl, alkenyl, alkynyl or aryl iodides are preferred as alkylating agents.

Preferred alkylating agents also comprise alkanyl, alkenyl or aryl groups selected from the group consisting of (1) C₂H₅; (2) C₃ to C₂₀ straight-chain, branched or cyclic alkane or alkene; (3) C₃ to C₂₀ straight-chain, branched or cyclic alkane or alkene comprising a heteroatom selected from the group consisting of O, N and S; (4) unsubstituted or substituted C₆ to C₂₀ aromatic, wherein the substituent is selected from the group consisting of (a) C₁ to C₁₂ straight-chain, branched or cyclic alkane or alkene, optionally substituted with Cl, Br or F, and (b) a halogen selected from the group consisting of Cl, Br and F; and (5) unsubstituted or substituted C₆ to C₂₀ aromatic comprising a heteroatom selected from the group consisting of O, N and S, wherein the substituent is selected from the group consisting of (a) C₁ to C₁₂ straight-chain, branched or cyclic alkane or alkene, optionally substituted with Cl, Br or F, and (b) a halogen selected from the group consisting of Cl, Br and F.

In another embodiment, preferred alkylating agents comprise alkanyl and alkenyl groups selected from the group consisting of (1) C₂H₅, (2) C₃ to C₁₂ straight-chain, branched or cyclic alkane or alkene, and (3) C₃ to C₁₂ straight-chain, branched or cyclic alkane or alkene comprising a heteroatom selected from the group consisting of O, N and S. In another embodiment, preferred aryl groups are unsubstituted or substituted phenyl, wherein the substituent is selected from the group consisting of (a) C₁ to C₁₂ straight-chain, branched or cyclic alkane or alkene, optionally substituted with Cl, Br or F, and (b) a halogen selected from the group consisting of Cl, Br and F.

The solvent used in the N-alkylation reaction must be anhydrous and may be any suitable anhydrous solvent, such as tetrahydrofuran (THF), ethyl ether, dimethoxyethyl ether or dioxane.

The alkylation reaction is quenched by the addition of about 10% aqueous sodium bisulfite and the reaction mixture is extracted with dichloromethane and dried over anhydrous sodium sulfate. Removal of the solvent under reduced pressure yields the crude N-substituted nepetalactam product, which may be purified by column chromatography on silica gel using ethyl acetate/hexanes as eluant. Fractions are monitored by thin layer chromatography (TLC) using 25% ethyl acetate/hexanes as eluant. This standard technique is described by Still, Kahn and Mitra [J. Org. Chem. (1978) 43:2923-2 925].

Fractions obtained by column chromatography containing the N-substituted dihydronepetalactams may be combined and solvent removed under reduced pressure to yield the purified N-substituted dihydronepetalactam products. The products may be analyzed by ¹H and ¹³C NMR techniques to verify structural identity.

N-Aryl dihydronepetalactams may also be prepared according to the method described by Chan, [Tetrahedron Letters (1996) 37:9013-9016] by reacting dihydronepetalactam with an appropriate triaryl bismuthane (Formula VI in Reaction VI) in the presence of Cu(OAc)₂ and triethylamine to form the N-aryl dihydronepetalactam (Formula VII in Reaction VI) 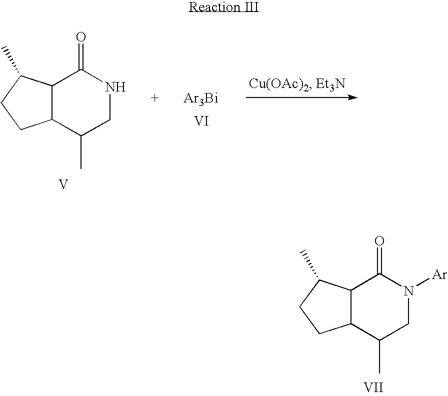
wherein Ar is an unsubstituted or substituted aromatic group as defined above for Formula IV.

In view of the structures IIa-IId as shown above, the compounds described herein will be recognized as exhibiting stereoisomerism, both enantiomerism and diastereomerism as the case may be. Unless a specific stereoisomer is indicated, the discussion will be understood to refer to all possible isomers, whether the structures are shown in the stereochemically ambiguous form of the structure of Formula IV, or are shown as a specific stereoisomer when other stereoisomers are also possible.

A compound according to this invention includes a compound that is a single stereoisomer as well as a compound that is a mixture of stereoisomers. A composition may be formed from a mixture of the compounds of this invention in which R, as described above, differs among the various compounds from which the composition is formed.

Dihydronepetalactam, N-methyl dihydronepetalactam and the compounds described by Formula IV are all compounds that may be used for a multiplicity of purposes, such as use as an active in an effective amount for the repellency of various insect or arthropod species, or as a fragrance compound in a perfume composition, or as a topical treatment for skin. For example, these compounds may be applied in a topical manner to the skin, hide, hair, fur or feathers of a human or animal host for an insect or arthropod, or to an inanimate host such as growing plants or crops, to impart insect or arthropod repellency or a pleasant odor or aroma. An inanimate host may also include any article of manufacture that is affected by insects, such as buildings, furniture and the like. Typically, these articles of are considered to be insect-acceptable food sources or insect-acceptable habitats.

A repellent or repellent composition refers to a compound or composition that drives insects or arthropods away from their preferred hosts or from insect-suitable articles of manufacture. Most known repellents are not active poisons at all, but rather prevent damage to humans, animals plants and/or articles of manufacture by making insect/arthropod food sources or living conditions unattractive or offensive. Typically, a repellent is a compound or composition that can be topically applied to a host, or can be incorporated into an insect susceptible article, to deter insects/arthropods from approaching or remaining in the nearby 3-dimensional space in which the host or article exists. In either case, the effect of the repellent is to drive the insects/arthropods away from, or to reject, (1) the host, thereby minimizing the frequency of “bites” to the host, or (2) the article, thereby protecting the article from insect damage. Repellents may be in the form of gases (olfactory), liquids, or solids (gustatory).

One property that is important to overall repellent effectiveness is surface activity, as many repellents contain both polar and non-polar regions in their structure. A second property is volatility. Repellents form an unusual class of compounds where evaporation of the active ingredient from the host's skin surface, or from an insect-repellent article, is necessary for effectiveness, as measured by the protection of the host from bites or the protection of the article from damage.

In the case of a topical insect/arthropod repellent applied to the skin, hide, hair, feathers or fur of a host, an aspect of the potency of the repellent is the extent to which the concentration of the repellent in the air space directly above the surface where applied is sufficient to repel the insects/arthropods. A desirable level of concentration of the repellent is obtained in the air space primarily from evaporation, but the rate of evaporation is affected by the rate absorption into the skin or other surface, and penetration into and through the surface is thus almost always an undesirable mode of loss of repellent from the surface. Similar considerations must be made for articles that contain a repellent, or into which a repellent has been incorporated, as a minimum concentration of repellent is required in the three-dimensional air space surrounding the article itself to obtain the desired level of protection.

In selecting a substance for use as an insect/arthropod repellent active, the inherent volatility is thus an important consideration. A variety of strategies are available, however, when needed for the purpose of attempting to increase persistence of the active while not decreasing, and preferably increasing, volatility. For example, the active can be formulated with polymers and inert ingredients to increase persistence on a surface to which applied or within an article. The presence of inert ingredients in the formulation, however, dilutes the active in the formulation as applied, and the loss from undesirably rapid evaporation must thus be balanced against the risk of simply applying too little active to be effective. Alternatively, the active ingredient may be contained in microcapsules to control the rate of loss from a surface or an article; a precursor molecule, which slowly disintegrates on a surface or in an article, may be used to control the rate of release the active ingredient; or a synergist may be used to continually stimulate the evaporation of the active from the composition.

The release of the active ingredient may be accomplished, for example, by sub-micron encapsulation, in which the active ingredient is encapsulated (surrounded) within a skin nourishing protein just the way air is captured within a balloon. The protein may be used, for example, at about a 20% concentration. An application of repellent contains many of these protein capsules that are suspended in either a water-based lotion, or water for spray application. After contact with skin, the protein capsules begin to breakdown releasing the encapsulated active. The process continues as each microscopic capsule is depleted then replaced in succession by a new capsule that contacts the skin and releases its active ingredient. The process may take up to 24 hours for one application. Because a protein adheres very effectively to skin, these formulations are very resistant to perspiration (sweat-off) and water from other sources.

One of the distinct advantages of dihydronepetalactam, N-methyl dihydronepetalactam and the compounds described by Formula IV is that they are all characterized by a relative volatility that makes them suitable for use to obtain a desirably high level of concentration of active on, above and around a surface or article, as described above. One or more of these dihydronepetalactam compounds are typically used for such purposes as an active in a composition in which the compounds are admixed with a carrier suitable for wet or dry application of the composition to any surface in the form, for example, of a liquid, aerosol, gel, aerogel, foam or powder (such as a sprayable powder or a dusting powder). Suitable carriers include any one of a variety of commercially available organic and inorganic liquid, solid, or semi-solid carriers or carrier formulations usable in formulating skin or insect repellent products. When formulating a skin product or topical insect repellent, it is preferred to select a dermatologically acceptable carrier. For example the carrier may include water, alcohol, silicone, petrolatum, lanolin or many of several other well known carrier components. Examples of organic liquid carriers include liquid aliphatic hydrocarbons (e.g. pentane, hexane, heptane, nonane, decane and their analogs) and liquid aromatic hydrocarbons.

Examples of other liquid hydrocarbons include oils produced by the distillation of coal and the distillation of various types and grades of petrochemical stocks, including kerosene oils that are obtained by fractional distillation of petroleum. Other petroleum oils include those generally referred to as agricultural spray oils (e.g. the so-called light and medium spray oils, consisting of middle fractions in the distillation of petroleum and which are only slightly volatile). Such oils are usually highly refined and may contain only minute amounts of unsaturated compounds. Such oils, moreover, are generally paraffin oils and accordingly can be emulsified with water and an emulsifier, diluted to lower concentrations, and used as sprays. Tall oils, obtained from sulfate digestion of wood pulp, like the paraffin oils, can similarly be used. Other organic liquid carriers can include liquid terpene hydrocarbons and terpene alcohols such as alpha-pinene, dipentene, terpineol, and the like.

Other carriers include silicone, petrolatum, lanolin, liquid hydrocarbons, agricultural spray oils, paraffin oil, tall oils, liquid terpene hydrocarbons and terpene alcohols, aliphatic and aromatic alcohols, esters, aldehydes, ketones, mineral oil, higher alcohols, finely divided organic and inorganic solid materials. In addition to the above-mentioned liquid hydrocarbons, the carrier can contain conventional emulsifying agents which can be used for causing the nepetalactam compound to be dispersed in, and diluted with, water for end-use application. Still other liquid carriers can include organic solvents such as aliphatic and aromatic alcohols, esters, aldehydes, and ketones. Aliphatic monohydric alcohols include methyl, ethyl, normal-propyl, isopropyl, normal-butyl, sec-butyl, and tert-butyl alcohols. Suitable alcohols include glycols (such as ethylene and propylene glycol) and pinacols. Suitable polyhydroxy alcohols include glycerol, arabitol, erythritol, sorbitol, and the like. Finally, suitable cyclic alcohols include cyclopentyl and cyclohexyl alcohols.

Conventional aromatic and aliphatic esters, aldehydes and ketones can be used as carriers, and occasionally are used in combination with the above-mentioned alcohols. Still other liquid carriers include relatively high-boiling petroleum products such as mineral oil and higher alcohols (such as cetyl alcohol). Additionally, conventional or so-called “stabilizers” (e.g. tert-butyl sulfinyl dimethyl dithiocarbonate) can be used in conjunction with, or as a component of, the carrier or carriers used in a composition as made according to this invention.

Numerous clays having a layered structure with interstices, and synthetic inorganic materials that resemble such clays in respect of chemical composition, crystallinity and layered morphology, are suitable for use herein as carriers. Suitable clays having a layered structure with interstices include smectite, kaolin, muscovite, vermiculite, phlogopite, xanthophyllite, and chrysotile, and mixtures thereof. Preferred are smectite clays and kaolin clays. Smectite clays include montmorillonite, beidellite, nontronite, saponite, hectorite, sauconite, and others. Kaolin clays include kaolinite, deckite, nacrite, antigorite, and others. Most preferred is montmorillonite. Average particle sizes range from 0.5 to 50 micrometers.

Desirable properties of a topical composition or article repellent to insects and/or arthropods include low toxicity, resistance to loss by water immersion or sweating, low or no odor or at least a pleasant odor, ease of application, and rapid formation of a dry tack-free surface film on the host's skin or other surface. In order to obtain these properties, the formulation for a topical repellent or repellant article should permit animals infested with insects and/or arthropods (e.g. dogs with fleas, poultry with lice, cows with horn flies or ticks, and humans) to be treated with a repellent (including a composition thereof) by contacting the skin, hide, hair, fur or feathers of such human or animal with an effective amount of the repellent for repelling the insect or arthropod from the human or animal host.

The application of an effective amount of an repellant composition on a surface subject to attack by insects (such as skin, hide, hair, fur, feathers or plant or crop surface) may be accomplished by dispersing the repellent into the air or dispersing the repellent as a liquid mist or incorporated into a powder or dust, and this will permit the repellent to fall on the desired host surfaces. It may also be desirable to formulate a repellent by combining a dihydronepetalactam compound to form a composition with a fugitive vehicle for application in the form of a spray. Such a composition may be an aerosol, sprayable liquid or sprayable powder composition adapted to disperse the active compound into the atmosphere by means of a compressed gas, or a mechanical pump spray. Likewise, directly spreading of a liquid/semi-solid/solid repellent on the host in wet or dry form (as a friable solid, for example) is an effective method of contacting the surface of the host with an effective amount of the repellent.

Further, it may also be desirable to combine one or more of the active compounds described herein with one or more other compounds known to have insect repellency in a composition to achieve the synergistic effect as may result from such a combination. Suitable compounds known for insect repellency combinable for such purpose include but are not limited to dihydronepetalactone, benzil, benzyl benzoate, 2,3,4,5-bis(butyl-2-ene)tetrahydrofurfural, butoxypolypropylene glycol, N-butylacetanilide, normal-butyl-6,6-dimethyl-5,6-dihydro-1,4-pyrone-2-carboxylate, dibutyl adipate, dibutyl phthalate, di-normal-butyl succinate, N,N-diethyl-meta-toluamide, dimethyl carbate, dimethyl phthalate, 2-ethyl-2-butyl-1,3-propanediol, 2-ethyl-1,3-hexanediol, di-normal-propyl isocinchomeronate, 2-phenylcyclohexanol, p-methane-3,8-diol, and normal-propyl N,N-diethylsuccinamate.

In addition to one or more of the active compounds described herein, an insect repellent composition may also include one or more essential oils and/or active ingredients of essential oils. “Essential oils” are defined as any class of volatile oils obtained from plants possessing the odor and other characteristic properties of the plant. Examples of useful essential oils include: almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, and oil of wintergreen. Examples of active ingredients in essential oils are: citronellal, methyl salicylate, ethyl salicylate, propyl salicylate, citronellol, safrole, and limonene.

The insects and arthropods that may be repelled by the compounds and/or compositions of this invention may include any member of a large group of invertebrate animals characterized, in the adult state (non-adult insect states include larva and pupa) by division of the body into head, thorax, and abdomen, three pairs of legs, and, often (but not always) two pairs of membranous wings. This definition therefore includes a variety of biting insects (e.g. ants, bees, chiggers, fleas, mosquitoes, ticks, wasps), biting flies [e.g. black flies, green head flies, stable flies, horn flies (haematobia irritans)], wood-boring insects (e.g. termites), noxious insects (e.g. houseflies, cockroaches, lice, roaches, wood lice), and household pests (e.g. flour and bean beetles, dust mites, moths, silverfish, weevils).

A host from which it may be desired to repel an insect may include any plant or animal (including humans) affected by insects. Typically, hosts are considered to be insect-acceptable food sources or insect-acceptable habitats. For example, humans and animals serve as food source hosts for blood-feeding insects and arthropods such as biting flies, chiggers, fleas, mosquitoes, ticks and lice.

In another embodiment, a dihydronepetalactam compound may be used as a fragrance compound or as an active in a fragrance composition, and be applied in a topical manner to human or animal skin or hair to impart a pleasing fragrance, as in skin lotions and perfumes for humans or pets.

Particularly because of the pleasant aroma associated with the compounds hereof, a further embodiment of this invention is one in which one or more dihydronepetalactam compounds are formulated into a composition for use as a product that is directed to other fundamental purposes. The fragrance and/or insect repellency of these products will be enhanced by the presence therein of an active compound or composition of this invention. Such products include without limitation colognes, lotions, sprays, creams, gels, ointments, bath and shower gels, foam products (e.g. shaving foams), makeup, deodorants, shampoo, hair lacquers/hair rinses, and personal soap compositions (e.g. hand soaps and bath/shower soaps). The compound(s) may of course be incorporated into such products simply to impart a pleasing aroma. Any means of incorporation such as is practiced in the art is satisfactory.

A corresponding aspect of the wide variety of products discussed above is a further alternative embodiment of this invention, which is a process for fabricating a composition of matter, a topical treatment for skin, or an article of manufacture, by providing as the composition, or incorporating into the composition, skin treatment or article, one or more dihydronepetalactam compounds, or a mixture of stereoisomers thereof. Such products, and the method and process described above, illustrate the use of a dihydronepetalactam compound as a fragrance compound or perfume, or in a fragrance composition or formulation, or in a topical treatment for skin, or in an article of manufacture. In fabricating a composition of matter, for example, the composition could be prepared as a sprayable liquid, an aerosol, a foam, a cream, an ointment, a gel, a paste, a powder or a friable solid. The process of fabrication in such case would thus include admixing an active with suitable carriers or other inert ingredients to facilitate delivery in the physical form as described, such as liquid carriers that are readily sprayed; a propellant for an aerosol or a foam; viscous carriers for a cream, an ointment, a gel or a paste; or dry or semi-solid carriers for a powder or a friable solid.

A composition containing one or more of the above described active compounds prepared as an insect/arthropod repellent, fragrance product, skin treatment or other personal care product may also contain other therapeutically or cosmetically active adjuvants or supplemental ingredients as are typical in the personal care industry. Examples of these include fungicides, sunscreening agents, sunblocking agents, vitamins, tanning agents, plant extracts, anti-inflammatory agents, anti-oxidants, radical scavenging agents, retinoids, alpha-hydroxy acids, antiseptics, antibiotics, antibacterial agents, antihistamines; adjuvants such as thickeners, buffering agents, chelating agents, preservatives, gelling agents, stabilizers, surfactants, emolients, coloring agents, aloe vera, waxes, and penetration enhancers; and mixtures of any two or more thereof.

In a further embodiment of this invention, a dihydronepetalactam compound is incorporated into an article to produce an insect/arthropod repellent effect. Articles contemplated to fall within this embodiment include manufactured goods, including textile goods such as clothing, outdoor or military equipment as mosquito netting, natural products such as lumber, or the leaves of insect vulnerable plants.

In another embodiment of this invention, a dihydronepetalactam compound is incorporated into an article to produce a fragrance pleasing to humans, or a nepetalactam compound is applied to the surface of an object to impart an odor thereto. The particular manner of application will depend upon the surface in question and the concentration required to impart the necessary intensity of odor. Articles contemplated to fall within these embodiments include manufactured goods, including textile goods, air fresheners, candles, various scented articles, fibers, sheets, paper, paint, ink, clay, wood, furniture (e.g. for patios and decks), carpets, sanitary goods, plastics, polymers, and the like.

A dihydronepetalactam compound may be admixed in a composition with other components, such as a carrier, in an amount that is effective for usage for a particular purpose, such as an insect/arthropod repellant, fragrance or other skin treatment. The amount of the active compound contained in a composition will generally not exceed about 80% by weight based on the weight of the final product, however, greater amounts may be utilized in certain applications, and this amount is not limiting. More preferably, a suitable amount of the compound will be at least about 0.001% by weight and preferably about 0.01% up to about 50% by weight; and more preferably, from about 0.01% to about 20% weight percent, based on the total weight of the total composition or article. Specific compositions will depend on the intended use.

Other methods of using a dihydronepetalactam are as disclosed in US 2003/062,357; US 2003/079,786; and US 2003/191,047, each of which is incorporated in its entirety as a part hereof.

The present invention is further described in, but not limited by, the following specific embodiments.

### EXAMPLES

### General Procedures

All reactions and manipulations related to the synthesis of the control and test repellents were carried out in a standard laboratory fume hood in standard laboratory glassware. Nepetalactone (II), consisting mainly of the cis, trans-stereoisomer, was obtained by steam distillation of commercially-available catnip oil from Nepeta cataria, obtained from Berjé, (Bloomfield, N.J.). All inorganic salts and organic solvents, with the exception of anhydrous THF, were obtained from VWR Scientific (West Chester, Pa.). All other reagents used in the examples were obtained from Sigma-Aldrich Chemical (Milwaukee, Wis.) and used as received. Determination of pH was done with pHydrion paper from Micro Essential Laboratory, Inc. (Brooklyn, N.Y.). The lactam products were purified by column chromatography on silica gel using ethyl acetate/hexanes as the eluant; the purified products were characterized by NMR spectroscopy. NMR spectra were obtained on a Bruker DRX Advance (500 MHz ¹H, 125 MHz ¹³C; Bruker Biospin Corp., Billerica, Mass.) using deuterated solvents obtained from Cambridge Isotope Laboratories, Inc. (Andover, Mass.).

The meaning of abbreviations used is as follows: “mL” means milliliter(s), “μL” means microliter, “g” means gram(s), “mg” means milligram, “psi” means pounds per square inch, “MP” means melting point, “NMR” means nuclear magnetic resonance, “° C.” means degrees Centigrade, and “ATP” means adenosine triphosphate.

### Synthesis of tris(4-chlorophenyl)bismuthane (a triaryl bismuthane used for Reaction VI)

To a solution of 100 mL of 1M 4-chlorophenyl magnesium bromide in diethyl ether cooled in an ice bath under nitrogen was added dropwise a solution of 10.51 g bismuth trichloride in 50 mL of tetrahydrofuran so as to maintain the temperature below 5° C. The reaction was allowed to warm to room temperature and was stirred for an additional 1 hr. A solution of 50 mL of saturated aqueous ammonium chloride was added at 5° C. to quench the reaction. The solid from the reaction was removed by filtration and extracted with 200 mL of diethyl ether. The combined filtrate was washed with 100 mL of saturated aqueous ammonium chloride. The ammonium chloride solution was extracted with 200 mL of diethyl ether, and the combined ether solution was washed two times with 75 mL of saturated aqueous ammonium chloride. The ether solution was dried over anhydrous magnesium sulfate and concentrated in vacuo to give a crude solid, which was extracted with several portions of hot hexane. The hexane extracts (400 mL) were combined and concentrated in vacuo to give the tris(4-chlorophenyl)bismuthane as a yellow solid (13.94 g, 62% yield, m.p. 100° C.). NMR analysis of the product was consistent with that of tris(4-chlorophenyl)bismuthane.

### Synthesis tris(4-bromophenyl)bismuthane (a triaryl bismuthane used for Reaction VI)

To a solution of 320 mL of 4-bromophenyl magnesium bromide in diethyl ether (prepared by reacting 54.9 g of 1,4-dibromobenzene and 5.63 g of magnesium) cooled in an ice bath under nitrogen was added dropwise a solution of 23.6 g bismuth trichloride in 120 mL of tetrahydrofuran over 1 hr, maintaining the temperature below 7° C. The reaction was allowed to warm to room temperature and was stirred for an additional 1 hr. A solution of 60 mL of saturated aqueous ammonium chloride was added at 5° C. to quench the reaction. The solid from the reaction was removed by filtration and extracted with 150 mL of diethyl ether. The aqueous layer was extracted three times with 100 mL of diethyl ether. The combined ether solution was washed with 150 mL of saturated aqueous ammonium chloride and dried over anhydrous magnesium sulfate and concentrated in vacuo to give a crude solid, which was extracted with several portions of hot hexane. The hexane extracts (700 mL) were combined and concentrated in vacuo to give the tris(4-bromophenyl)bismuthane as a yellow solid (17.5 g, 35% yield, m.p. 112° C.). NMR analysis of the product was consistent with that of tris(4-bromophenyl)bismuthane.

The procedures described in Examples 1 through 15 were used to synthesize the compounds shown in Table 1, where structure number refers to the dihydronepetalactam derivative substituted with the indicated R-group.

TABLE 1
N-substituted dihydronepetalactams

| | structure |
|---|---|
| R | number |
| H | V |
| methyl | IVa |
| ethyl | IVb |
| n-propyl | IVc |
| n-butyl | IVd |
| n-pentyl | IVe |
| n-hexyl | IVf |
| n-octyl | IVg |
| i-propyl | IVh |
| allyl | IVi |
| propargyl | IVj |
| phenyl | VIIa |
| p-chlorophenyl | VIIb |
| p-bromophenyl | VIIc |

### Example 1

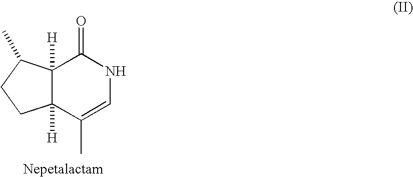

### (4aS,7S,7aR)-4,7-dimethyl-2,4a,5,6,7,7a-hexahydro-1H-cyclopenta[c]pyridin-1-one

Nepetalactam was prepared from cis, trans-nepetalactone according to the method of Eisenbraun, et al. (supra). In a 1 liter reaction vessel, 100 g of cis,trans-neptalactone in 250 mL of dichloromethane, along with a Teflon®-coated stirring bar, was sealed with a pressure regulator. The vessel was evacuated under vacuum and filled with gaseous ammonia three times and then charged with ammonia to 103.4 kPa. The solution was stirred under constant pressure of ammonia at room temperature for three days. The vessel was vented and purged with nitrogen. The solution was transferred to a 500 mL round-bottomed flask and the solvent was removed under reduced pressure to yield a thick yellow syrup (109.49 g). The crude nepetalactam was purified by vacuum distillation to a pale yellow crystalline solid. Recrystallization of the solid from hexanes yielded pure nepetalactam (89.60 g, 88% yield) with an observed MP=94-96° C. (literature MP=95-96° C.)

### Example 2

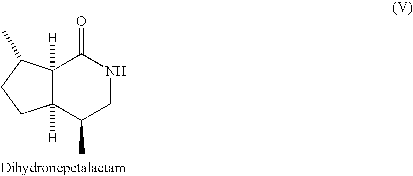

### (4S,4aR,7S,7aR)-4,7-dimethyloctahydro-1H-cyclopenta[c]pyridin-1-one

In a 100 mL pressure reaction vessel 10 g of nepetalactam in 20 mL of 95% ethanol was treated with 0.25 g of 2% Pd/SrCO₃. The vessel was sealed with a pressure regulator and then evacuated under vacuum and filled with hydrogen seven times. The vessel was then charged to a pressure of 103.4 kPa with hydrogen and was stirred under constant pressure of hydrogen at room temperature for three days. The vessel was vented and purged with nitrogen. The mixture was then filtered through a bed of celite, rinsing with 50 mL additional ethanol. Removal of the solvent from the filtrate yielded 10.24 g of dihydronepetalactam as a clear oil that solidified on standing to a low melting solid. NMR analysis of the product obtained was consistent with the dihydronepetalactam structure depicted in structural representation V.

### Example 3

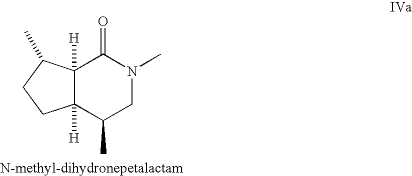

### (4S,4aR,7S,7aR)-2,4,7-trimethyloctahydro-1H-cyclopenta[c]pyridin-1-one

In a 100 mL round-bottomed flask, 1.0 g of dihydronepetalactam IV in 30 mL of THF was treated with 2.15 g of iodomethane, 0.85 g of potassium hydroxide and 0.39 g of tetrabutylammonium bromide at room temperature with stirring. After three days, the solvent was removed from the reaction under reduced pressure. Water (50 mL) was added to the resulting residue and the aqueous mixture was extracted with 25 mL of dichloromethane three times. The combined organic layers were dried over anhydrous sodium sulfate and the solvent was removed under reduced pressure to yield the N-methyl-dihydronepetalactam IVa as a yellow oil, 0.69 g (63% yield). The product was purified by column chromatography on silica gel eluting with ethyl acetate/hexanes. NMR analysis of the product obtained was consistent with the N-methyl-dihydronepetalactam structure depicted in structural representation IVa.

### Example 4

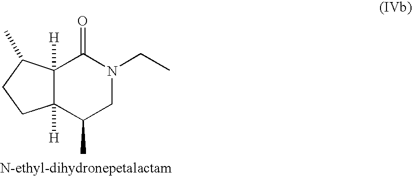

### (4S,4aR,7S,7aR)-2-ethyl-4,7-dimethyloctahydro-1H-cyclopenta[c]pyridin-1-one

An oven-dried, 250 mL three-necked round-bottomed flask was cooled to room temperature under a stream of nitrogen; a solution of 1.68 g of dihydronepetalactam (V) in 30 mL of dry THF was added via pipette and cooled with ice bath to 0° C. Separately, 0.80 g of 30% potassium hydride-mineral oil suspension was washed with 10 mL of hexanes three times to remove the mineral oil. The resulting white solid was added in small portions to the reaction solution while stirring at 0° C., resulting in gas evolution. After the addition was complete, the reaction mixture was stirred for 30 minutes, treated with 1.2 mL of iodoethane and then allowed to stir at 0° C. for 30 minutes. The reaction was then warmed to room temperature for 30 minutes and quenched by the addition of 30 mL of a 10% aqueous solution of sodium bisulfite. The mixture was extracted with 20 mL of dichloromethane three times and the combined organics were dried over anhydrous sodium sulfate. Removal of the solvent under reduced pressure afforded the crude product as a yellow oil, which was purified by column chromatography on silica gel eluting with ethyl acetate/hexanes. Purified product (0.75 g, 38% yield) was obtained, and NMR analysis was consistent with the N-ethyl-dihydronepetalactam structure depicted in structural representation IVb.

### Example 5

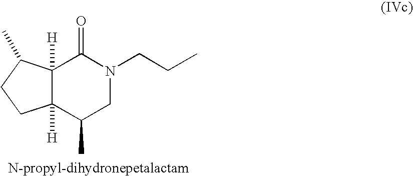

### (4S,4aR,7S,7aR)-2-n-propyl-4,7-dimethyloctahydro-1H-cyclopenta[c]pyridin-1-one

An oven-dried, 250 mL three-necked round-bottomed flask was cooled to room temperature under a stream of nitrogen; a solution of 1.12 g of dihydronepetalactam (V) in 30 mL of dry THF was added via pipette and cooled with ice bath to 0° C. Separately, 0.90 g of 30% potassium hydride-mineral oil suspension was washed with 10 mL of hexanes three times to remove the mineral oil. The resulting white solid was added in small portions to the reaction solution while stirring at 0° C., resulting in gas evolution. After the addition was complete, the reaction mixture was stirred for 30 minutes, treated with 1.46 mL of iodopropane and then allowed to stir at 0° C. for 30 minutes. The reaction was then warmed to room temperature for 30 minutes and quenched by the addition of 30 mL of a 10% aqueous solution of sodium bisulfite. The mixture was extracted with 20 mL of dichloromethane three times and the combined organics were dried over anhydrous sodium sulfate. Removal of the solvent under reduced pressure afforded the crude product as a yellow oil, which was purified by column chromatography on silica gel eluting with ethyl acetate/hexanes. Purified product (1.41 g, 67% yield) was obtained, and NMR analysis was consistent with the N-propyl-dihydronepetalactam structure depicted in structural representation IVc.

### Example 6

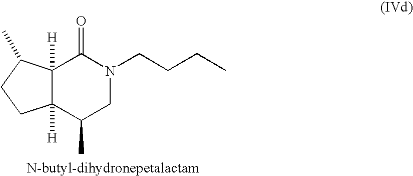

### (4S,4aR,7S,7aR)-2-n-butyl-4,7-dimethyloctahydro-1H-cyclopenta[c]pyridin-1-one

An oven-dried, 250 mL three-necked round-bottomed flask was cooled to room temperature under a stream of nitrogen; a solution of 1.12 g of dihydronepetalactam (V) in 30 mL of dry THF was added via pipette and cooled in an ice bath to 0° C. Separately, 0.80 g of 30% potassium hydride-mineral oil suspension was washed with 10 mL of hexanes three times to remove the mineral oil. The resulting white solid was added in small portions to the reaction solution while stirring at 0° C. resulting in gas evolution. After the addition was complete, the reaction mixture stirred for 30 minutes, treated with 1.67 mL of iodobutane and then allowed to stir at 0° C. for 30 minutes. The reaction was then warmed to room temperature for 30 minutes and quenched by the addition of 30 mL of a 10% aqueous solution of sodium bisulfite. The mixture was extracted with 20 mL of dichloromethane three times and the combined organics were dried over anhydrous sodium sulfate. Removal of the solvent under reduced pressure afforded the crude product as a yellow oil which was purified by column chromatography on silica gel eluting with ethyl acetate/hexanes. Purified product (0.89 g, 60% yield) was obtained, and NMR analysis was consistent with the N-butyl-dihydronepetalactam structure depicted in structural representation IVd.

### Example 7

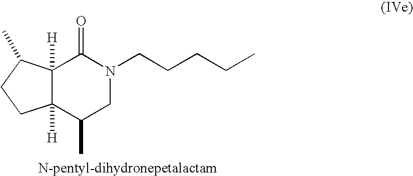

### (4S,4aR,7S,7aR)-2-n-pentyl-4,7-dimethyloctahydro-1H-cyclopenta[c]pyridin-1-one

An oven-dried, 250 mL three-necked round-bottomed flask was cooled to room temperature under a stream of nitrogen; a solution of 4.65 g of nepetalactam (II) in 100 mL of dry THF was added to the flask via pipette while the flask was being purged with nitrogen, and the solution was cooled in an ice bath to 0° C. under nitrogen. Separately, 6.05 g of 30% potassium hydride-mineral oil suspension was washed with 30 mL of hexanes three times to remove the mineral oil. The resulting white solid was added in small portions to the reaction solution with stirring at 0° C., resulting in gas evolution. After the addition was complete, the reaction mixture was stirred for 30 minutes, treated with 5.93 mL of iodopentane and then allowed to stir at 0° C. for 30 minutes. The reaction was then warmed to room temperature for 30 minutes and quenched by the addition of 50 mL of a saturated aqueous solution of sodium bisulfite. The mixture was extracted with 30 mL of dichloromethane three times and the combined organics were dried over 10% sodium bisulfite. Removal of the solvent under reduced pressure afforded the crude product (7.2 g) as a brown oil, which was purified by column chromatography on silica gel using ethyl acetate/hexanes as the eluant to yield purified product (4.4 g, 67% yield). NMR analysis of the purified product was consistent with N-pentyl-nepetalactam.

In a 250 mL pressure reaction vessel, 2.4 g of N-pentyl-nepetalactam in 100 mL of 95% ethanol was treated with 0.70 g of 2% Pd/SrCO₃. The vessel was sealed with a pressure regulator and then evacuated under vacuum and filled with hydrogen three times. The vessel was then charged to a pressure of 103.4 kPa with hydrogen and was stirred under constant pressure of hydrogen at room temperature overnight. The vessel was vented and purged with nitrogen. The mixture was then filtered through a bed of celite, rinsing with 50 mL additional ethanol. Removal of the solvent under reduced pressure afforded the crude product as a yellow oil which was purified by column chromatography on silica gel eluting with ethyl acetate/hexanes. Purified product (1.5 g, 62% yield) was obtained, and NMR analysis was consistent with the N-pentyl-dihydronepetalactam structure depicted in structural representation IVe.

### Example 8

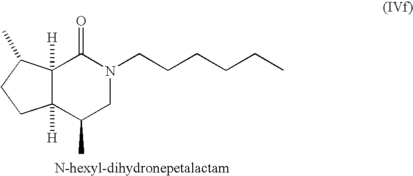

### (4S,4aR,7S,7aR)-2-n-hexyl-4,7-dimethyloctahydro-1H-cyclopenta[c]pyridin-1-one

An oven-dried, 250 mL three-necked round-bottomed flask was cooled to room temperature under a stream of nitrogen; a solution of 4.65 g of nepetalactam (II) in 100 mL of dry THF was added to the flask via pipette while the flask was being purged with nitrogen, and the solution was cooled in an ice bath to 0° C. under nitrogen. Separately, 6.0 g of 30% potassium hydride-mineral oil suspension was washed with 30 mL of hexanes three times to remove the mineral oil. The resulting white solid was added in small portions to the reaction solution with stirring at 0° C., resulting in gas evolution. After the addition was complete, the reaction mixture was stirred for 30 minutes, treated with 6.7 mL of iodohexane and then allowed to stir at 0° C. for 30 minutes. The reaction was then warmed to room temperature for 30 minutes and quenched by the addition of 30 mL of a 10% aqueous solution of sodium bisulfite. The mixture was extracted with 30 mL of dichloromethane three times and the combined organics were dried over anhydrous sodium sulfate. Removal of the solvent under reduced pressure afforded the crude product (5.46 g) as a brown oil, which was purified by column chromatography on silica gel using ethyl acetate/hexanes as the eluant to yield purified product (3.2 g, 46% yield). NMR analysis of the purified product was consistent with N-hexyl-nepetalactam.

In a 250 mL pressure reaction vessel, 1.5 g of N-hexyl-nepetalactam in 100 mL of 95% ethanol was treated with 0.70 g of 2% Pd/Sr/CO₃. The vessel was sealed with a pressure regulator and then evacuated under vacuum and filled with hydrogen three times. The vessel was then charged to a pressure of 103.4 kPa with hydrogen and was stirred under constant pressure of hydrogen at room temperature overnight. The vessel was vented and purged with nitrogen. The mixture was then filtered through a bed of celite, rinsing with 50 mL additional ethanol. Removal of the solvent under reduced pressure afforded the crude product as a yellow oil, which was purified by column chromatography on silica gel eluting with ethyl acetate/hexanes. Purified product (1.35 g, 89% yield) was obtained, and NMR analysis was consistent with the N-hexy-dihydronepetalactam structure depicted in structural representation IVf.

### Example 9

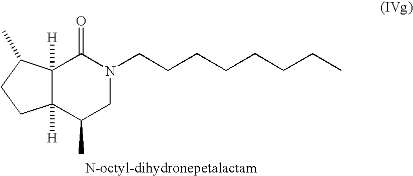

### (4S,4aR,7S,7aR)-2-n-octyl-4,7-dimethyloctahydro-1H-cyclopenta[c]pyridin-1-one

An oven-dried, 250 mL three-necked round-bottomed flask was cooled to room temperature under a stream of nitrogen; a solution of 4.65 g of nepetalactam (II) in 30 mL of dry THF was added to the flask via pipette while the flask was being purged with nitrogen, and the solution was cooled in an ice bath to 0° C. under nitrogen. Separately, 6.0 g of 30% potassium hydride-mineral oil suspension was washed with 30 mL of hexanes three times to remove the mineral oil. The resulting white solid was added in small portions to the reaction solution with stirring at 0° C., resulting in gas evolution. After the addition was complete, the reaction mixture was stirred for 30 minutes, treated with 8.2 mL of iodooctane and then allowed to stir at 0° C. for 30 minutes. The reaction was then warmed to room temperature for 30 minutes and quenched by the addition of 30 mL of a 10% aqueous solution of sodium bisulfite. The mixture was extracted with 30 mL of dichloromethane three times and the combined organics were dried over anhydrous sodium sulfate. Removal of the solvent under reduced pressure afforded the crude product (5.36 g) as a brown oil, which was purified by column chromatography on silica gel using ethyl acetate/hexanes as the eluant to yield purified product (4.26 g, 53% yield). NMR analysis of the purified product was consistent with N-octyl-nepetalactam.

In a 250 mL pressure reaction vessel, 2.4 g of N-octyl-nepetalactam in 100 mL of 95% ethanol was treated with 0.70 g of 2% Pd/Sr/CO₃. The vessel was sealed with a pressure regulator and then evacuated under vacuum and filled with hydrogen three times. The vessel was then charged to a pressure of 103.4 kPa with hydrogen and was stirred under constant pressure of hydrogen at room temperature overnight. The vessel was vented and purged with nitrogen. The mixture was then filtered through a bed of celite, rinsing with 50 mL additional ethanol. Removal of the solvent under reduced pressure afforded the crude product as a yellow oil, which was purified by column chromatography on silica gel eluting with ethyl acetate/hexanes. Purified product (1.7 g, 68% yield) was obtained, and NMR analysis was consistent with the N-octyl-dihydronepetalactam structure depicted in structural representation IVg.

### Example 10

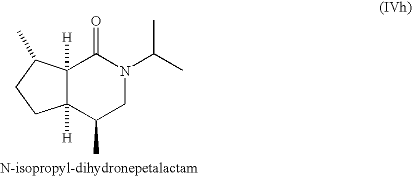

### (4S,4aR,7S,7aR)-2-n-isopropyl-4,7-dimethyloctahydro-1H-cyclopenta[c]pyridin-1-one

An oven-dried, 250 mL three-necked round-bottomed flask was cooled to room temperature under a stream of nitrogen; a solution of 3.0 g of nepetalactam (II) in 50 mL of dry THF was added via pipette and cooled in an ice bath to 0° C. Separately, 4.0 g of 30% potassium hydride-mineral oil suspension was washed with 10 mL of hexanes three times to remove the mineral oil. The resulting white solid was added in small portions to the reaction solution while stirring at 0° C., resulting in gas evolution. After the addition was complete, the reaction mixture stirred for 30 minutes, treated with 5.0 g of 2-iodopropane and then allowed to stir at 0° C. for 30 minutes. The reaction was then warmed to room temperature for 30 minutes and quenched by the addition of 30 mL of a 10% aqueous solution of sodium bisulfite. The mixture was extracted with 20 mL of dichloromethane three times and the combined organics were dried over anhydrous sodium sulfate. Removal of the solvent under reduced pressure afforded the crude product as a yellow oil, which was purified by column chromatography on silica gel eluting with ethyl acetate/hexanes. Purified product (3.0 g, 85% yield) was obtained, and NMR analysis was consistent with N-isopropyl-nepetalactam.

In a 250 mL pressure reaction vessel, 2.4 g of N-isopropyl-nepetalactam in 100 mL of 95% ethanol was treated with 0.70 g of 2% Pd/Sr/CO₃. The vessel was sealed with a pressure regulator and then evacuated under vacuum and filled with hydrogen three times. The vessel was then charged to a pressure of 103.4 kPa with hydrogen and was stirred under constant pressure of hydrogen at room temperature overnight. The vessel was vented and purged with nitrogen. The mixture was then filtered through a bed of celite, rinsing with 50 mL additional ethanol. Removal of the solvent under reduced pressure afforded the crude product as a yellow oil, which was purified by column chromatography on silica gel eluting with ethyl acetate/hexanes. Purified product (0.96 g, 40% yield) was obtained, and NMR analysis was consistent with N-isopropyl-dihydronepetalactam structure depicted in structural representation IVh.

### Example 11

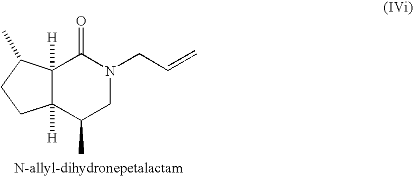

### (4S,4aR,7S,7aR)-2-n-allyl-4,7-dimethyloctahydro-1H-cyclopenta[c]pyridin-1-one

An oven-dried, 250 mL three-necked round-bottomed flask was cooled to room temperature under a stream of nitrogen; a solution of 0.93 g of dihydronepetalactam (V) in 20 mL of dry THF was added via pipette and cooled in an ice bath to 0° C. Separately, 1.9 of 30% potassium hydride-mineral oil suspension was washed with 10 mL of hexanes three times to remove the mineral oil. The resulting white solid was added in small portions to the reaction solution while stirring at 0° C., resulting in gas evolution. After the addition was complete, the reaction mixture stirred for 30 minutes, treated with 1.52 mL of allyl iodide and then allowed to stir at 0° C. for 30 minutes. The reaction was then warmed to room temperature for 30 minutes and quenched by the addition of 30 mL of a 10% aqueous solution of sodium bisulfite. The mixture was extracted with 20 mL of dichloromethane three times and the combined organics were dried over anhydrous sodium sulfate. Removal of the solvent under reduced pressure afforded the crude product as a yellow oil, which was purified by column chromatography on silica gel eluting with ethyl acetate/hexanes. Purified product (0.503 g, 43% yield) was obtained, and NMR analysis was consistent with the N-allyl-dihydronepetalactam structure depicted in structural representation IVi.

### Example 12

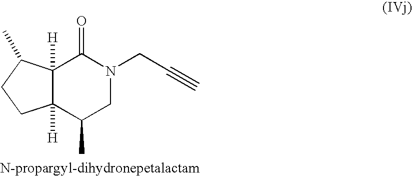

### (4S,4aR,7S,7aR)-2-n-propargyl-4,7-dimethyloctahydro-1H-cyclopenta[c]pyridin-1-one

An oven-dried, 250 mL three-necked round-bottomed flask was cooled to room temperature under a stream of nitrogen; a solution of 1.00 g of dihydronepetalactam (V) in 30 mL of dry THF was added via pipette and cooled in an ice bath to 0° C. Separately, 1.2 g of 30% potassium hydride-mineral oil suspension was washed with 10 mL of hexanes three times to remove the mineral oil. The resulting white solid was added in small portions to the reaction solution while stirring at 0° C., resulting in gas evolution. After the addition was complete, the reaction mixture was stirred for 30 minutes, treated with 1.07 g of propargyl bromide and then allowed to stir at 0° C. for 30 minutes. The reaction was then warmed to room temperature for 30 minutes and quenched by the addition of 30 mL of a saturated 10% solution of sodium bisulfite. The mixture was extracted with 20 mL of dichloromethane three times and the combined organics were dried over anhydrous sodium sulfate. Removal of the solvent under reduced pressure afforded the crude product as a yellow oil, which was purified by column chromatography on silica gel eluting with ethyl acetate/hexanes. Purified product (0.64 g, 52% yield) was obtained, and NMR analysis was consistent with the N-propargyl-dihydronepetalactam structure depicted in structural representation IVj.

### Example 13

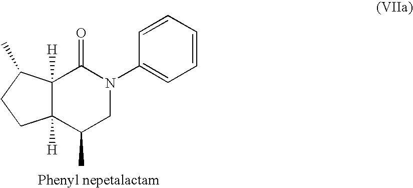

### (4S,4aR,7S,7aR)-4,7-dimethyl-2-phenyloctahydro-1H-cyclopenta[c]pyridin-1-one

A slurry of 0.25 g dihydronepetalactam (V), 1.31 g of triphenylbismuthane, 0.27 g of anhydrous copper(II) acetate, 0.41 mL of triethylamine in 10 mL of dichloromethane was stirred at room temperature for 24 hours. Removal of the solvent under reduced pressure afforded the crude reaction mixture, which was purified by column chromatography on silica gel using ethyl acetate/hexanes as the eluant to yield purified product as a white solid (0.23 g, 63% yield). NMR analysis of the purified product was consistent with the N-phenyl-dihydronepetalactam structure depicted in structural representation VIIa.

### Example 14

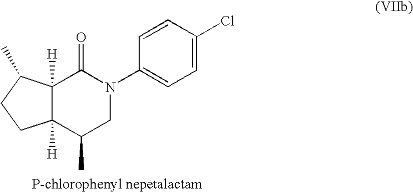

### (4S,4aR,7S,7aR)-2-(4-chlorophenyl)-4,7-dimethyloctahydro-1H-cyclopenta[c]pyridin-1-one

A slurry of 0.15 g nepetalactam (V), 0.98 g of tris(4-chlorophenyl)bismuthane, 0.16 g of anhydrous copper(II) acetate, 0.25 mL of triethylamine in 15 mL of dichloromethane was stirred at room temperature for 24 hours. Removal of the solvent under reduced pressure afforded the crude reaction mixture, which was purified by column chromatography on silica gel using ethyl acetate/hexanes as the eluant to yield purified product as an off-white solid (0.16 g, 64% yield). NMR analysis of the purified product was consistent with the N-4-chlorophenyl-dihydronepetalactam structure depicted in structural representation VIIb.

### Example 15

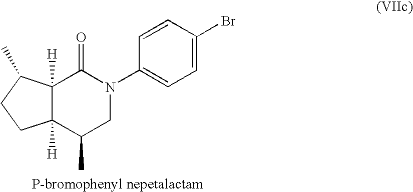

### (4S,4aR,7S,7aR)-2-(4-bromophenyl)-4,7-dimethyloctahydro-1H-cyclopenta[c]pyridin-1-one

A slurry of 0.15 g nepetalactam (II), 1.22 g of tris(4-bromophenyl)bismuthane, 0.16 g of anhydrous copper(II) acetate, 0.25 mL of triethylamine in 15 mL of dichloromethane was stirred at room temperature for 24 hours. Removal of the solvent under reduced pressure afforded the crude reaction mixture, which was purified by column chromatography on silica gel using ethyl acetate/hexanes as the eluant to yield a crude product as a colorless oil (0.08 g). The reaction was repeated at 4× the scale with identical procedure. The crude reaction mixture was combined with the crude product from the first run and purified by column chromatography on silica gel using ethyl acetate/hexanes as the eluant to yield the purified product as a colorless oil (0.16 g, 11% yield overall). NMR analysis of the purified product was consistent with the N-4-bromophenyl-dihydronepetalactam structure depicted in structural representation VIIc.

The products of Examples 1-15 were evaluated for insect repellency against Aedes aegypti mosqutioes in the in vitro Gupta box landing assay. In this method a chamber contained 5 wells, each covered by a Baudruche (animal intestine) membrane. Each well was filled with bovine blood containing sodium citrate (to prevent clotting) and ATP (72 mg ATP disodium salt per 26 ml of blood), and heated to 37° C. A volume of 25 μL of isopropyl alcohol (IPA) containing one test specimen or control was applied to each membrane. The concentrations of the dihydronepetalactam products were 1% (w/v) in IPA. The negative control was neat IPA and the positive control was a 1% (w/v) solution of DEET.

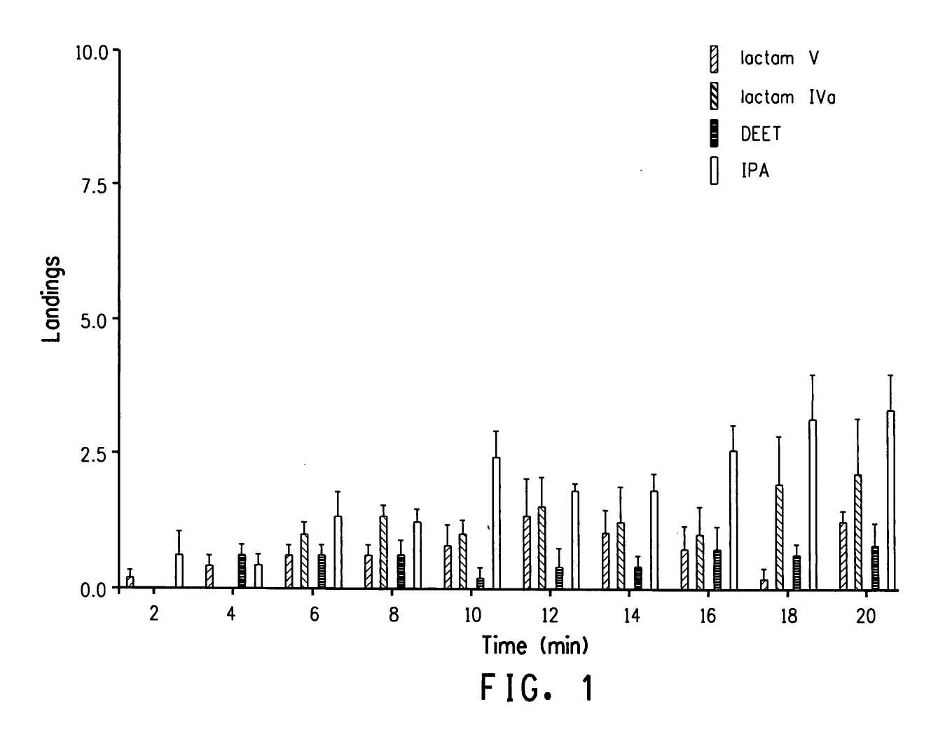

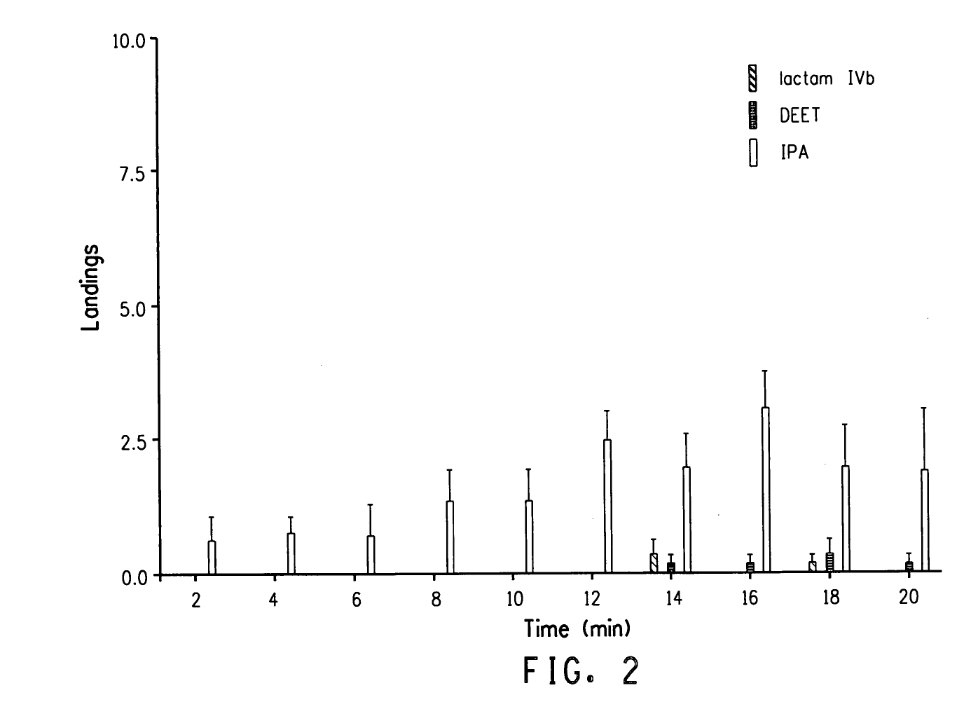

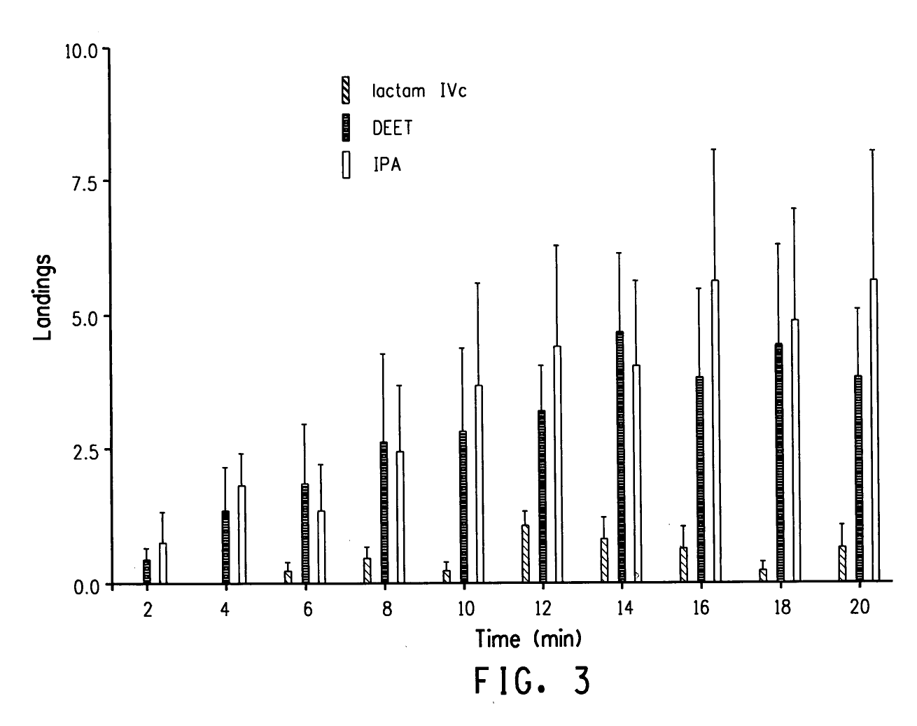

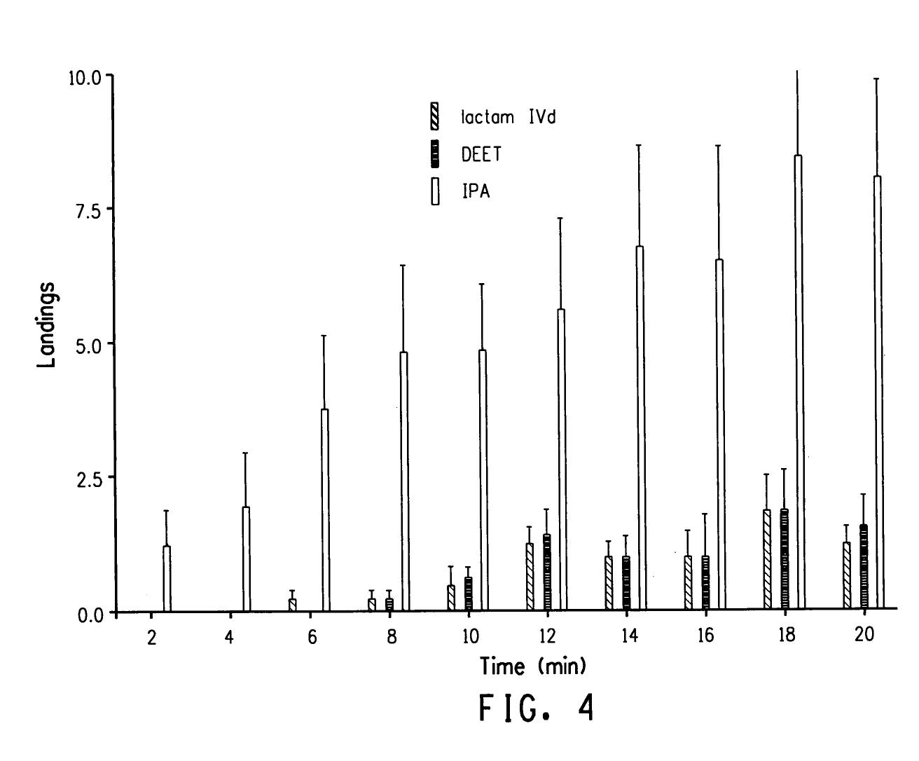

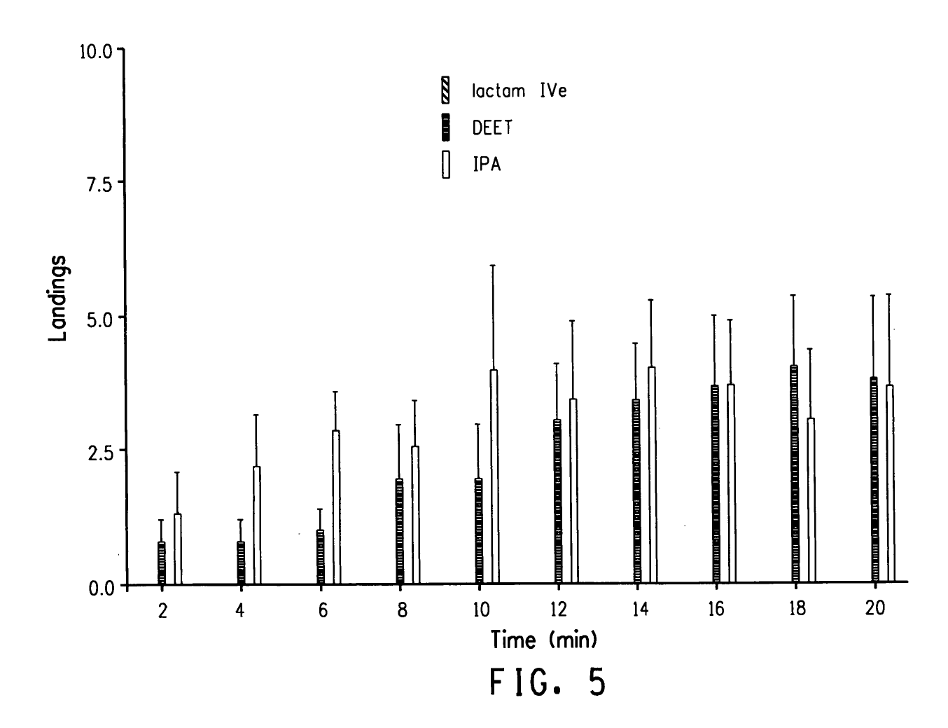

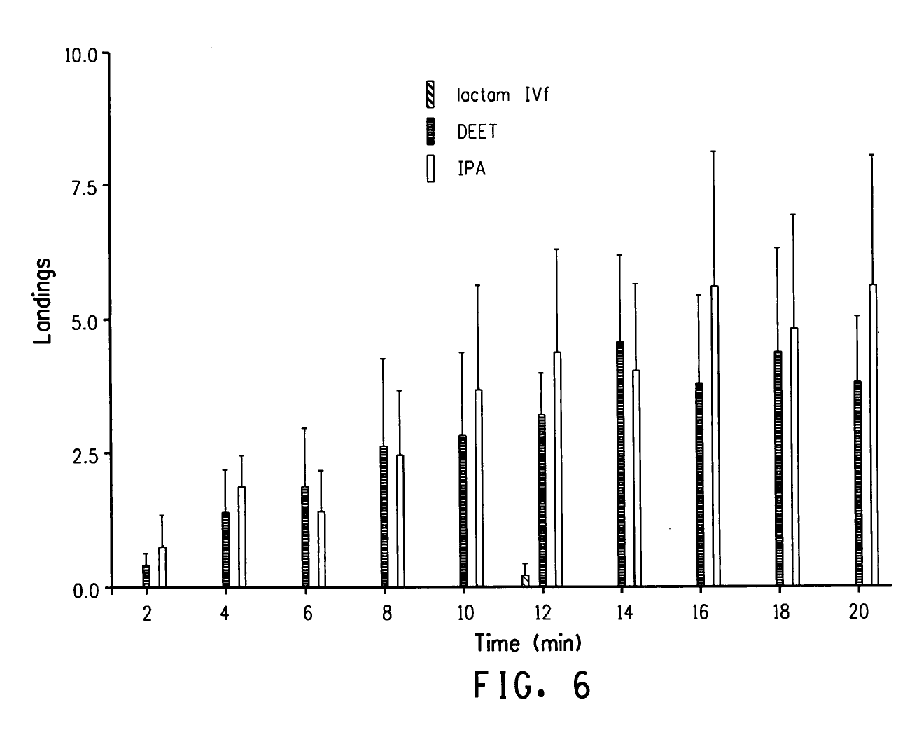

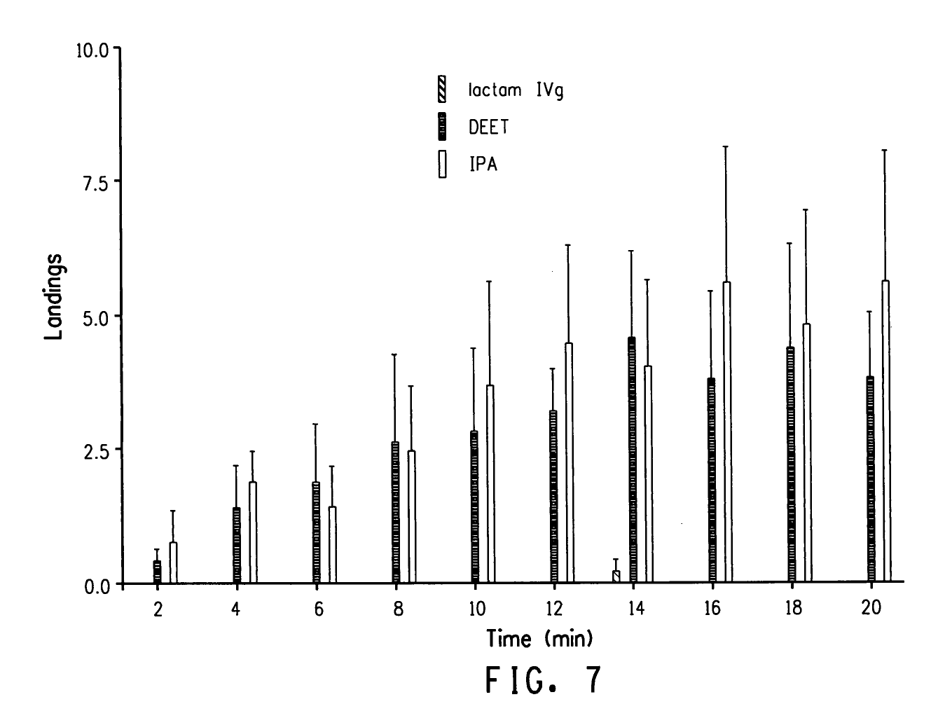

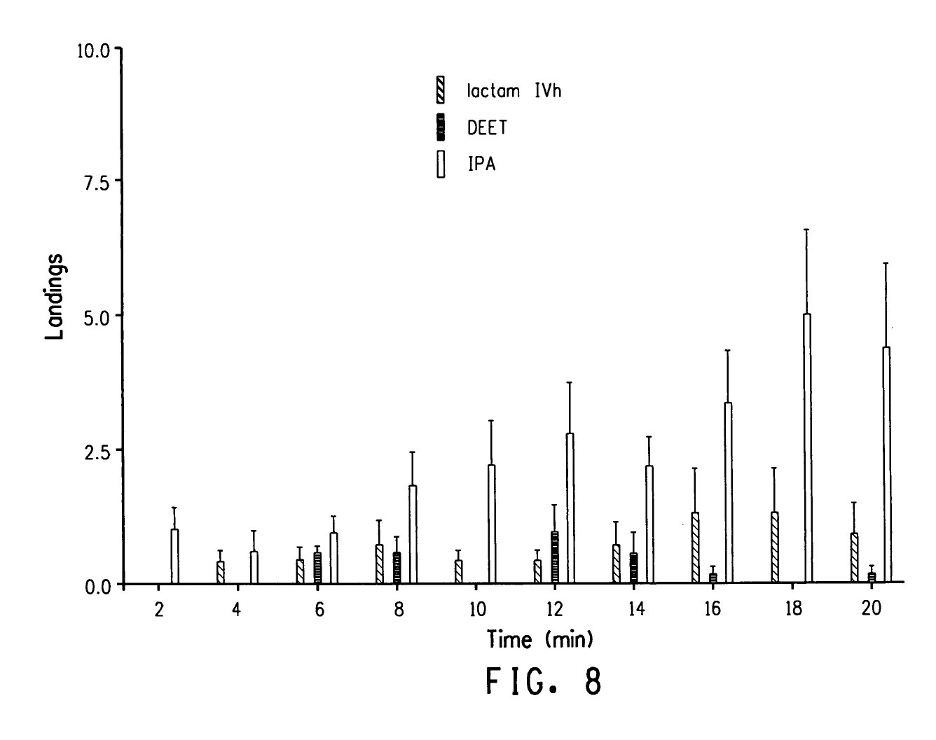

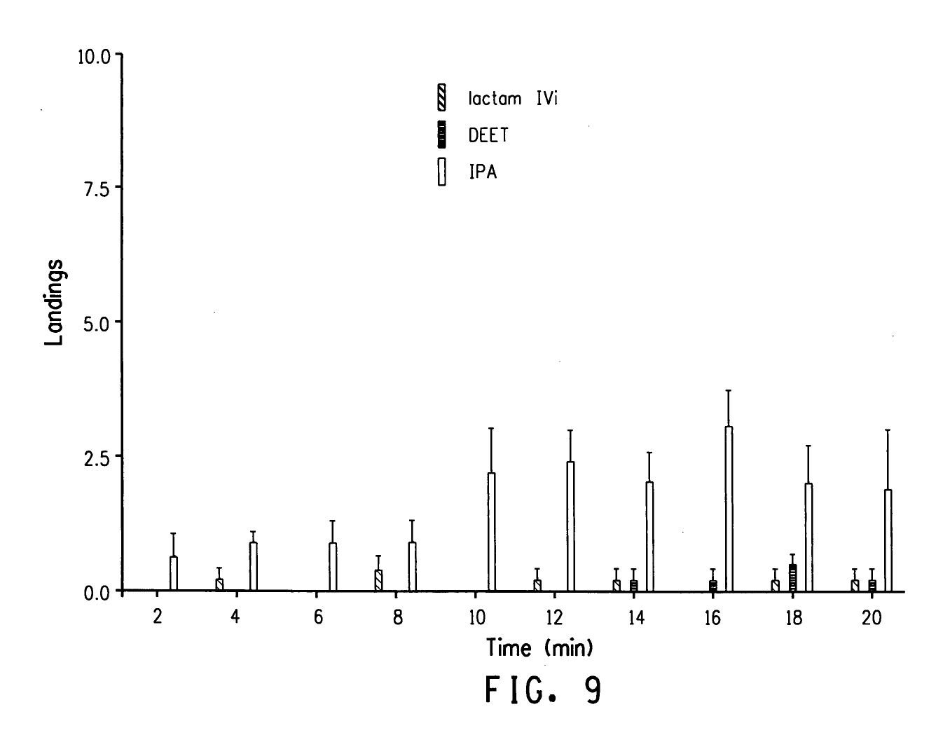

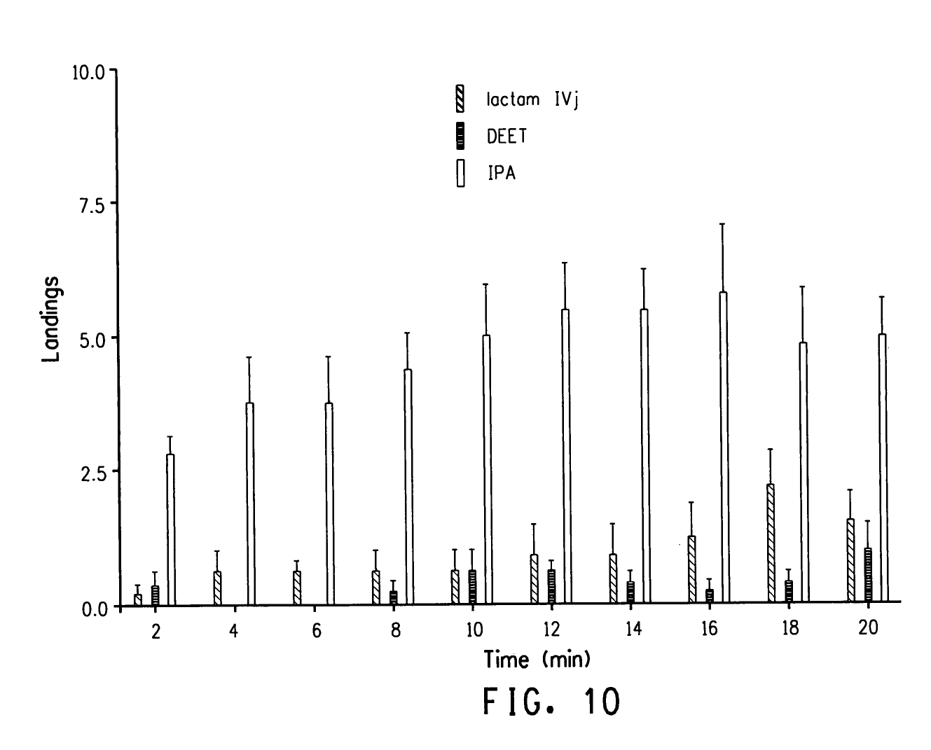

After 5 min, approximately 250 4-day-old female Aedes aegypti mosquitoes were introduced into the chamber. The number of mosquitoes probing the membranes for each treatment was recorded at 2 min intervals over 20 min. The results obtained in this manner with respect to the compounds of Examples 1˜12 are depicted in FIGS. 1-10 (labeled as Examples 16˜25), wherein each datum represents the mean of five replicate experiments.

From these data, the % mean repellency for a repellent at a given concentration of repellent test solution was determined using the following equation:
% mean repellency=C−T/C×100

where C=the total number of landings on the IPA control well, and T=the total number of landings on the test solution well. The % mean repellencies at 1% (w/v) are depicted in Table 2 for the compounds of Examples 1˜15, wherein R refers to the substituent on dihydronepetalactam.

TABLE 2
N-substituted dihydronepetalactams:
% mean repellencies at 1% (w/v)

| | structure | % mean |
|---|---|---|
| R | number | repellency |
| H | V | 69.6 |
| methyl | IVa | 39.6 |
| ethyl | IVb | 96.8 |
| n-propyl | Ivc | 87.9 |
| n-butyl | Ivd | 86.3 |
| n-pentyl | IVe | 100 |
| n-hexyl | IVf | 99.8 |
| n-octyl | IVg | 98.3 |
| i-propyl | IVh | 69.4 |
| allyl | IVi | 90.6 |
| propargyl | IVj | 88.1 |
| phenyl | VIIa | 59.5 |
| p-chlorophenyl | VIIb | 81.5 |
| p-bromophenyl | VIIc | 54.1 |

What is claimed is:

1. A compound represented schematically by the following formula: 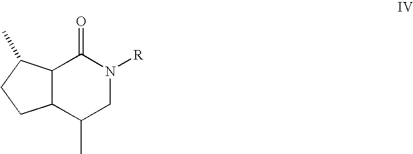
wherein R comprises (a) an alkane radical other than methyl, (b) an alkene radical, (c) an alkyne radical, or (d) an aromatic radical.

2. The compound of claim 1 wherein R comprises (a) C₂ to C₂₀ alkane, (b) C₂ to C₂₀ alkene, (c) C₃ to C₂₀ alkyne, or (d) C₆ to C₂₀ aromatic.

3. The compound of claim 1 wherein R comprises a member of the group consisting of:
(a) C₂H₅,
(b) C₃ to C₂₀ straight-chain, branched or cyclic alkane or alkene,
(c) C₃ to C₂₀ straight-chain, branched or cyclic alkane or alkene comprising a heteroatom selected from the group consisting of O, N and S,
(d) unsubstituted or substituted C₆ to C₂₀ aromatic, wherein the substituent is selected from the group consisting of (i) C₁ to C₁₂ straight-chain, branched or cyclic alkane or alkene, optionally substituted with Cl, Br or F, and (ii) a halogen selected from the group consisting of Cl, Br and F, and
(e) unsubstituted or substituted C₆ to C₂₀ aromatic comprising a heteroatom selected from the group consisting of O, N and S, wherein the substituent is selected from the group consisting of (i) C₁ to C₁₂ straight-chain, branched or cyclic alkane or alkene, optionally substituted with Cl, Br or F, and (ii) a halogen selected from the group consisting of Cl, Br and F.

4. The compound of claim 1 wherein R is selected from the group consisting of (a) C₂H₅, (b) C₃ to C₁₂ straight-chain, branched or cyclic alkane and alkene, and (c) C₃ to C₁₂ straight-chain, branched or cyclic alkane or alkene comprising a heteroatom selected from the group consisting of O, N and S.

5. The compound of claim 1 wherein R is unsubstituted or substituted phenyl, wherein the substituent is selected from the group consisting of (a) C₁ to C₁₂ straight-chain, branched or cyclic alkane or alkene, optionally substituted with Cl, Br or F, and (b) a halogen selected from the group consisting of Cl, Br and F.

6. The compound of claim 1 which is a single stereoisomer of a single compound, or is a mixture of stereoisomers of a single compound.

7. A composition of matter comprising (a) a carrier, and (b) a compound described generally by the following formula: 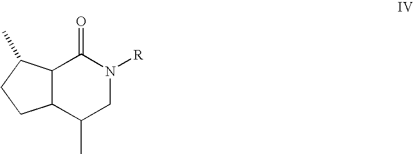
wherein R is H, an alkane radical, an alkene radical, an alkyne radical, or an aromatic radical.

8. The composition of claim 7 wherein R is (a) H, (b) C₁ to C₂₀ alkane, (c) C₂ to C₂₀ alkene, (d) C₃ to C₂₀ alkyne, or (e) C₆ to C₂₀ aromatic.

9. The composition of claim 7 wherein R is selected from the group consisting of:
(a) CH₃, C₂H₅,
(b) C₃ to C₂₀ straight-chain, branched or cyclic alkane or alkene,
(c) C₃ to C₂₀ straight-chain, branched or cyclic alkane or alkene comprising a heteroatom selected from the group consisting of O, N and S,
(d) unsubstituted or substituted C₆ to C₂₀ aromatic, wherein the substituent is selected from the group consisting of (i) C₁ to C₁₂ straight-chain, branched or cyclic alkane or alkene, optionally substituted with Cl, Br or F, and (ii) a halogen selected from the group consisting of Cl, Br and F, and
(e) unsubstituted or substituted C₆ to C₂₀ aromatic comprising a heteroatom selected from the group consisting of O, N and S, wherein the substituent is selected from the group consisting of (i) C₁ to C₁₂ straight-chain, branched or cyclic alkane or alkene, optionally substituted with Cl, Br or F, and (ii) a halogen selected from the group consisting of Cl, Br and F.

10. The composition of claim 7 wherein R is selected from the group consisting of (a) CH₃, (b) C₂H₅, (c) C₃ to C₁₂ straight-chain, branched or cyclic alkane and alkene, and (d) C₃ to C₁₂ straight-chain, branched or cyclic alkane or alkene comprising a heteroatom selected from the group consisting of O, N and S.

11. The composition of claim 7 wherein R is unsubstituted or substituted phenyl, wherein the substituent is selected from the group consisting of (a) C₁ to C₁₂ straight-chain, branched or cyclic alkane or alkene, optionally substituted with Cl, Br or F, and (b) a halogen selected from the group consisting of Cl, Br and F.

12. The composition of claim 7 which is a single stereoisomer of a single compound, or is a mixture of stereoisomers of a single compound.

13. The composition of claim 7 further comprising an insect repellent selected from the group consisting of dihydronepetalactone, benzil, benzyl benzoate, 2,3,4,5-bis(butyl-2-ene)tetrahydrofurfural, butoxypolypropylene glycol, N-butylacetanilide, normal-butyl-6,6-dimethyl-5,6-dihydro-1,4-pyrone-2-carboxylate, dibutyl adipate, dibutyl phthalate, di-normal-butyl succinate, N,N-diethyl-meta-toluamide, dimethyl carbate, dimethyl phthalate, 2-ethyl-2-butyl-1,3-propanediol, 2-ethyl-1,3-hexanediol, di-normal-propyl isocinchomeronate, 2-phenylcyclohexanol, p-methane-3,8-diol, and normal-propyl N,N-diethylsuccinamate.

14. The composition of claim 7 further comprising an essential oil.

15. The composition of claim 14 wherein the essential oil is selected from any one or more members of the group consisting of almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, and oil of wintergreen.

16. The composition of claim 7 further comprising any one or more members of the group of adjuvants consisting of a fungicide, sunscreening agent, sunblocking agent, vitamin, tanning agent, plant extract, anti-inflammatory agent, anti-oxidant, radical scavenging agent, retinoid, alpha-hydroxy acid, antiseptic, antibiotic, antibacterial agent, antihistamine.

17. The composition of claim 7 which comprises the compound in an amount of from about 0.001% to about 80% by weight of the total weight of the composition.

18. The composition of claim 7 in the form of a sprayable liquid, an aerosol, a foam, a cream, an ointment, a gel, a paste, a powder or a friable solid.

19. A method for repelling an insect or arthropod comprising exposing the insect or arthropod to a compound described generally by the following formula: 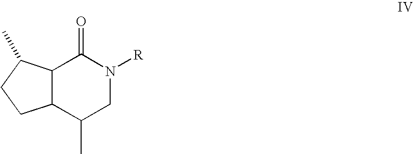
wherein R is H, an alkane radical, an alkene radical, an alkyne radical, or an aromatic radical.

20. The method of claim 19 wherein R is (a) H, (b) C₁ to C₂₀ alkane, (c) C₂ to C₂₀ alkene, (d) C₃ to C₂₀ alkyne, or (e) C₆ to C₂₀ aromatic.

21. The compound of claim 19 wherein R is selected from the group consisting of:
(a) CH₃, C₂H₅,
(b) C₃ to C₂₀ straight-chain, branched or cyclic alkane or alkene,
(c) C₃ to C₂₀ straight-chain, branched or cyclic alkane or alkene comprising a heteroatom selected from the group consisting of O, N and S,
(d) unsubstituted or substituted C₆ to C₂₀ aromatic, wherein the substituent is selected from the group consisting of (i) C₁ to C₁₂ straight-chain, branched or cyclic alkane or alkene, optionally substituted with Cl, Br or F, and (ii) a halogen selected from the group consisting of Cl, Br and F, and
(e) unsubstituted or substituted C₆ to C₂₀ aromatic comprising a heteroatom selected from the group consisting of O, N and S, wherein the substituent is selected from the group consisting of (i) C₁ to C₁₂ straight-chain, branched or cyclic alkane or alkene, optionally substituted with Cl, Br or F, and (ii) a halogen selected from the group consisting of Cl, Br and F.

22. The method of claim 19 wherein R is selected from the group consisting of (a) CH₃, (b) C₂H₅, (b) C₃ to C₁₂ straight-chain, branched or cyclic alkane and alkene, and (c) C₃ to C₁₂ straight-chain, branched or cyclic alkane or alkene comprising a heteroatom selected from the group consisting of O, N and S.

23. The method of claim 19 wherein R is unsubstituted or substituted phenyl, wherein the substituent is selected from the group consisting of (a) C₁ to C₁₂ straight-chain, branched or cyclic alkane or alkene, optionally substituted with Cl, Br or F, and (b) a halogen selected from the group consisting of Cl, Br and F.

24. The method of claim 19 wherein the compound is a single stereoisomer of a single compound, or is a mixture of stereoisomers of a single compound.

25. The method of claim 19 which comprises exposing the insect or arthropod to a composition that comprises the compound in an amount of from about 0.001% to about 80% by weight of the total weight of the composition.

26. The method of claim 19, which comprises exposing a blood-feeding insect or arthropod to the compound.

27. The method of claim 19, which comprises exposing an insect or arthropod, selected from the group consisting of biting flies, chiggers, fleas, mosquitoes, ticks and lice to the compound.

28. The method of claim 19 which comprises applying the compound to the skin, hide, hair, feathers or fur of a human or animal host for an insect or arthropod.
